(19) United States
(12) Patent Application Publication
(10) Pub. No.: US 2021/0207193 A1
(43) Pub. Date: Jul. 8, 2021

(54) IN VITRO METHOD FOR MONITORING THE PATHOGEN LOAD IN AN ANIMAL POPULATION

(71) Applicant: EVONIK OPERATIONS GMBH, Essen (DE)

(72) Inventors: Frank THIEMANN, Nottuln (DE); Monika FLÜGEL, Steinhagen (DE); Stefan PELZER, Gütersloh (DE); Michelle DARGATZ, Bielefeld (DE); Andreas KAPPEL, Glashütten (DE); Florian BÖHL, Neckargemünd (DE); Emeka Ignatius IGWE, München (DE); Petra HERZOG, Hamburg (DE); Jan-Hinnerk JARCK, Hamburg (DE)

(73) Assignee: EVONIK OPERATIONS GMBH, Essen (DE)

(21) Appl. No.: 17/059,431

(22) PCT Filed: May 27, 2019
(86) PCT No.: PCT/EP2019/063608

§ 371 Date: Nov. 28, 2020

(30) Foreign Application Priority Data
May 29, 2018 (EP) 18174689.2

(51) Int. Cl. C12Q 1/689; C12Q 1/70; C12Q 1/6806

(52) U.S. Cl. CPC C12Q 1/689; C12Q 1/6806; C12Q 1/701

(57) ABSTRACT

The present invention pertains to an in vitro method for monitoring the load of at least one pathogen in an avian population, the method comprising the following steps: collecting and pooling excremental sample material deriving from an avian population; homogenizing the pooled sample material obtained in step (a); diluting and optionally stabilizing the pooled sample material obtained in step (b) with aqueous buffer solution; lysing the cell material contained in the diluted sample material obtained in step (c); isolating nucleic acid material from the lysed sample material of step (d); detecting and quantifying at least one pathogen-specific target gene, or functional fragment thereof, contained in the nucleic acid isolate obtained in step (e); repeating steps (a) to (f) at consecutive points in time; and observing alterations in amount of the at least one pathogen specific target gene over time.

### FIELD OF THE INVENTION

The present invention relates to an in vitro method for monitoring the load of at least one pathogen in an animal population. More specifically, the present invention pertains to non-invasive method for monitoring alterations in the load of at least one pathogen in an animal population over time.

### BACKGROUND OF THE INVENTION

Avoidance of pathogen infections or pathogen contamination is critically important for the welfare and performance of livestock animals. Diseases caused by pathogenic bacterial or viral species or by pathogenic single-cell eukaryotes lead to high economic losses due to reduced weight gain, poor feed conversion efficiency, increased mortality rates and greater medication costs.

In order to be able to early detect and efficiently react to early indications or even manifestations of the diseases, there is an urgent need for a fast and reliable ante mortem method for monitoring the pathogen load in animal populations.

### SUMMARY OF THE INVENTION

The present invention provides an in vitro method for monitoring the load of at least one pathogen in an animal population, the method comprising the following steps:
(a) collecting and pooling excremental sample material deriving from an animal population;
(b) homogenizing the pooled sample material obtained in step (a);
(c) diluting and optionally stabilizing the pooled sample material obtained in step (b) with aqueous buffer solution;
(d) lysing the cell material contained in the diluted sample material obtained in step (c);
(e) isolating nucleic acid material from the lysed sample material of step (d);
(f) detecting and quantifying at least one pathogen-specific target gene, or functional fragment thereof, contained in the nucleic acid isolate obtained in step (e);
(g) repeating steps (a) to (f) at consecutive points in time; and
(h) observing alterations in amount of the at least one pathogen specific target gene over time.

A further aspect of the present invention is the use of the methods according to the present invention for determining the necessity feed interventions or medical interventions or, alternatively, for controlling the effectivity of feed interventions or medical interventions.

In addition, the present invention provides a method for obtaining a pooled excremental sample representing the total pathogen load in an animal population, the method comprising:
(a1) dividing the animal house or the area in which the animal population is kept in a grid pattern of uniform cells;
(a2) identifying at least one random sample collection site within the first cell and taking one first sample at said at least one sample collection site; and
(a3) sequentially collecting individual excremental samples in the remaining cells using the same relative sample collection sites within each cell;
and optionally
(a4) repeating steps (a2) and (a3) for at least one replicate sample.

### DETAILED DESCRIPTION OF THE INVENTION

The present invention features methods for monitoring the load of at least one pathogen load in animal populations. More specifically, the present invention pertains to an in vitro method for monitoring the load of at least one pathogen in an animal population, the method comprising the following steps:
(a) collecting and pooling excremental sample material deriving from an animal population;
(b) homogenizing the pooled sample material obtained in step (a);
(c) diluting and optionally stabilizing the pooled sample material obtained in step (b) with aqueous buffer solution;
(d) lysing the cell material contained in the diluted sample material obtained in step (c);
(e) isolating nucleic acid material from the lysed sample material of step (d);
(f) detecting and quantifying at least one pathogen-specific target gene, or functional fragment thereof, contained in the nucleic acid isolate obtained in step (e);
(g) repeating steps (a) to (f) at consecutive points in time; and
(h) observing alterations in amount of the at least one pathogen specific target gene over time.

The term “pathogen-specific target gene” refers to a gene being specific for a pathogenic species or sub-species.

In accordance with step (g), steps (a) to (f) are to be repeated at consecutive points in time. As an example, after initial determination of the amount of the at least one pathogen-specific marker gene in a pooled excremental sample, the amount of said at least one pathogen-specific marker gene is monitored in test samples collected and analyzed in a weekly, daily or hourly manner. In one embodiment, the pooled excremental samples are collected at consecutive days. Pooled excremental test samples may be collected and analyzed on a daily basis from birth to slaughter.

In one specific embodiment for poultry, a first pooled test sample is collected and analyzed during the initial growth phase (starter phase, day 5 to day 10), a second pooled test sample is collected and analyzed during the enhanced growth phase (day 11 to day 18) and, optionally, a third pooled test sample is collected and analyzed on a later stage.

In an alternative embodiment, a first pooled test sample is collected and analyzed in the initial growth phase and further pooled test samples are collected and analyzed e.g. on a daily basis during the enhanced growth phase, optionally until slaughter.

By “alteration” is meant an increase or a decrease in the amount of said at least one pathogen-specific target gene. An alteration may be as little as 1%, 2%, 3%, 4%, 5%, 10%, 20%, 30% or by 40%, 50%, 60%, or even by as much as 75%, 80%, 90% or 100%.

The amount of the at least one pathogen-specific target gene in the pooled excremental sample correlates with the overall load of the respective pathogen in the animal population. An increase in the amount of said at least one pathogen-specific target gene over time indicates the propagation or progression of pathogen load. Conversely, a decrease in the amount at least one pathogen-specific target gene over time indicates a regression of pathogen load (recovery/healing).

The above method is particularly suitable for performing ante mortem diagnoses under field conditions.

In the context of the present invention, the at least one pathogen is a pathogen related to an infection typically acquired during livestock productions. The at least one pathogen may be a pathogenic bacterial species, a pathogenic viral species and/or a pathogenic single-cell eukaryote.

Pathogenic bacterial species may be, for example, Clostridium perfringens, Campylobacter jejuni, Salmonella species, Avian pathogenic E. coli species, Mycoplasma spp., Mycobacterium avium, and/or Pasteurella multocida.

Pathogenic viral species may be, for example, African swine fever virus, Akabane virus, Australian bat lyssavirus, avian influenza virus, avian paramyxovirus, avian pox viruses, bluetongue virus, border disease virus, bovine ephemeral fever virus, bovine parainfluenca virus, bovine parvovirus, bovine viral diarrhoea virus, caprine arthritis encephalitis virus, chicken anemia virus, classical swine fever virus, equine herpesvirus, equine infectious anaemia virus, equine influenza virus, equine viral arteritis virus, foot and mouth disease virus, hendra virus, herpes virus, infectious bursal disease virus, Marek's disease virus, Newcastle disease virus, Nipah virus, Pestivirus, Rabies virus, Rift valley fever virus, rinderpest virus, salmon anaemia virus, swine fever virus, swine influenza virus, swine vesicular disease virus, vesicular exanthema virus, vesicular stomatitis virus and/or white spot disease virus.

Pathogenic single-cell eukaryotes are, for example, Eimeria acervulina, E. maxima, E. tenella, E. mitis, E. praecox, E. necatrix, E. brunetti, or Fagellates for example Histomonas meleagridis, Chochlosoma spp.

In accordance with the present invention, the load of the above-mentioned pathogens may be monitored individually or simultaneously, i.e. by way of multiplex testing.

As used herein, the term “animal population” refers to a group of animal individuals belonging to the same species. The animal population may for example be a group of pets or domestic animals as occurring in animal breeding, a group of farm animals as occurring in livestock production or in livestock breeding, or a group of wild-living animals or zoo animals.

In one embodiment, the animal population is an animal flock as occurring in livestock production processes. For example, the animal population or the animal flock can be an avian flock; a flock of sheep, goat or cattle, a flock of horses or a flock of pigs.

In one specific embodiment, the animal population is an avian population.

The animal population may be an avian flock. The avian flock according to the invention is preferably poultry. Preferred poultry according to the invention are chickens, turkeys, ducks and geese. The poultry can be optimized for producing young stock. This type of poultry is also referred to as parent and grandparent animals. Preferred parent and grandparent animals are, accordingly, (grand)parent broilers, (grand)parent ducks, (grand)parent turkeys and (grand)parent geese.

The poultry according to the invention can also be selected from fancy poultry and wild fowl. Preferred fancy poultry or wild fowl are peacocks, pheasants, partridges, guinea fowl, quails, capercailzies, goose, pigeons and swans. Further preferred poultry according to the invention are ostriches and parrots. Most preferred poultry according to the invention are broilers.

The excremental sample material may be selected from the group consisting of litter samples, liquid manure samples or samples of bodily excrements and solutions/suspensions thereof. In general, the term “litter” refers to a mixture of animal excrements with the bedding material. More specifically and in the context of the present invention, the term “litter” refers to mixtures from excremental droppings and bedding material as found in the pen, cage or slat. The term “liquid manure samples” refers to mixed excremental samples containing feces and urine.

In one embodiment, the pooled excremental sample material is feces. The pooled excremental sample material may, for example be pooled feces, in particular pooled feces deriving from an avian population/avian flock, such as pooled broiler feces.

In an embodiment, the pooled sample material of step (a) is a composite sample from randomly selected individual excremental samples; in particular, a composite sample from individual fecal samples derived from an avian population/avian flock, such as a broiler flock.

The excremental samples to be taken from a specific population is ideally taken at a discrete number of sites within the animal house in order to obtain a pooled sample being representative for the animal population as a whole.

The sample size (i.e. the number of excremental samples to be taken; each sample taken at a specific site within the animal house) has to be determined in view of the actual stocking density, i.e. with the actual number of animals belonging to the avian population to be tested.

The sample size may be calculated using the following formula:

$${n}_{0} = \frac{{Z}^{2} pq}{{e}^{2}}$$

wherein
n₀ is the sample size recommendation
Z is 1.96 for 95% confidence level
p is the estimated portion of the population with the attribute in question q is 1−p, and
e is the confidence interval expressed as decimal.

In general, a minimum of 80 to 100 individual excremental samples are sufficient for most livestock avian populations. Broilers are usually kept in flocks which can consist of >20000 birds in one house. As an example, for a broiler flock of 20000 animals, 96 individual samples are required for a confidence level of 95%.

The above formula is particularly suitable for determining the sample size required for large population.

For smaller populations, (<=100), the sample size recommendation n₀ as obtained with the above formula may be further adjusted in accordance with the following formula:

$$n = \frac{{n}_{0}}{1 + \frac{( {n}_{0} - 1 )}{N}}$$

wherein
N is the population size, and
n is the adjusted sample size.

For obtaining the pooled excremental sample material as required in step (a), several sampling methods may be used.

In one embodiment, the pooled excremental sample may be obtained by systematic grid sampling (systematic random sampling). For this method, the area in which the animal population is kept is divided in a grid pattern of uniform cells or sub-areas based on the desired number of individual excremental samples (i.e. the sample size). Then, a random sample collection site is identified within the first grid cell and a first sample is taken at said site. Finally, further samples are obtained from adjacent cells sequentially—e.g. in a serpentine, angular or zig-zag fashion—using the same relative location within each cell. A random starting point can be obtained with a dice or a random number generator.

The above process may optionally be repeated for replicate samples. That is, a new random position is established for the single collection point to be repeated in all of the cells. By analyzing replicate samples, variabilities in the estimate of the mean provided by the original samples may be determined.

Accordingly, the aforementioned methods may further comprise the following sub-steps:
(a1) dividing the animal house or the area in which the animal population is kept in a grid pattern of uniform cells;
(a2) identifying at least one random sample collection site within the first cell and taking one first sample at said at least one sample collection site; and
(a3) sequentially collecting individual excremental samples in the remaining cells using the same relative sample collection sites within each cell;
and optionally
(a4) repeating steps (a2) and (a3) for at least one replicate sample.

The sample size corresponds to the number of cells in the grid pattern in case one sample is to be taken per cell. In general, in case x samples are to be taken per cell, the sample size is the number of cells, divided by x.

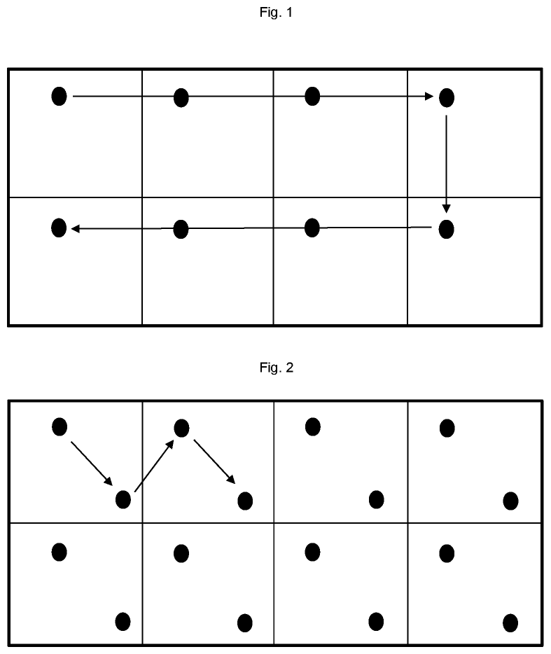

The systematic grid sampling method can be easily implemented in the field. Thereby, over- or underrepresentation of subareas can be avoided. Systematic grid sampling patterns according to the present invention are exemplified in FIG. 1 and FIG. 2.

Another sampling method is stratified random sampling (i.e. random sampling within a grid). Herein, samples are obtained sequentially from adjacent grid cells, but the location of the sample within each cell is random.

Alternatively, the samples may be taken by simple random sampling, where the samples are taken from random locations (without gridding) across the area in which the animals are kept. For this method, a formal approach for determining the random sample locations must be used, e.g. based upon a random number generator.

The samples may be collected manually with a spatula or a similar device and are immediately transferred into a sample collection vessel or tube.

In an alternative embodiment, the pooled excremental sample may be obtained using the overshoe method while walking through the house using a route that will produce representative samples for all parts of the house or the respective sector. Such route may e.g. be uniformly shaped serpentines or sinuous lines, angular lines or zigzag lines. Boot swabs being sufficiently absorptive to soak up moisture are particularly suitable. However, tube gauze socks are also acceptable.

Suitable sample volumes are, for example, 0.1 to 20 ml, in particular 0.2 to 10 ml, preferably 0.5 to 5 ml. Suitable sample masses are, for example, 0.1 to 20 g, in particular 0.2 to 10 g, preferably 0.5 to 5 g.

The sample material obtained in step (a) may comprise heterogeneous components such as used feed or litter material and thus has to be homogenized. The skilled artisan is aware of suitable, commonly used homogenization techniques.

The aqueous buffer solution used in step (c) for diluting and optionally stabilizing the pooled sample material obtained in step (b) comprises buffer solution, detergents, denaturing agent and/or complexing agents. Advantageously, said aqueous buffer solution comprises chaotropic salts to chemically disrupt cells and to stabilize and protect nucleic acids against nucleases in solution. Optionally, the aqueous buffer solution further comprises nuclease inhibitors.

The lysis step (d) of the method according to the present invention may be performed via chemical lysis or via mechanical lysis. Chemical lysis reagents are, for example, guanidiniumthiocyanat. Mechanical lysis may be accomplished by treating the sample with beads, ultrasonic or ultra Turrax®.

Advantageously, lysis step (d) according to the present invention comprises both, a mechanical lysis treatment and a chemical lysis treatment.

In one embodiment, lysis step (d) includes a heating step (d1), a grinding step (d2) and a spinning step (d3). For the heating step (d1), the diluted sample material is heated to 60° C. to 80° C., or to 65° C. to 75° C., for a time interval of between 10 min and 30 min, or between 15 min and 25 min. As an example, the diluted sample material may be heated for 20 min to 70° C. The grinding step (d2) involves treating the sample material with beads of 3 mm to 5 mm, depending on the volume of the sample vessel or tube. For example, the grinding step may be performed in a 50 ml tube using 4 to 7 beads being 4 mm in size. The spinning step (d3) may, for example, be performed by spinning the sample vessel or tube for 5 min at 2000×g.

In one embodiment, the nucleic acid isolation in step (e) is performed by magnetic bead extraction.

In an embodiment, the detection and quantification of the at least one pathogen-specific target gene in step (f) is performed via qPCR. For quantification, external calibrated quantification standards are used. Results are indicated as copies/μl (copies/g feces). The qPCR is performed on different time points of sample collection in the flock.

The qPCR-based detection method according to the present invention may include multiplex amplification of a plurality of markers or target genes simultaneously. It is well known in the art to select PCR primers to generate PCR products that do not overlap in size and can be analyzed simultaneously. Alternatively, it is possible to amplify different markers or target genes with primers that are differentially labeled and thus can be differentially detected.

The aforementioned methods may be used for determining the necessity feed interventions or medical interventions or, alternatively, for controlling the effectivity of feed interventions or medical interventions. An increase in the load of the at least one pathogen in an animal population may indicate the necessity of feed- or medical interventions. After intervening, the effectiveness of the intervention may be verified by the above methods for monitoring the load of the at least one pathogen in the animal population. In case the intervention is effective, a decrease in the load of the at least one pathogen is to be expected.

Feed interventions or medical interventions taken against the progression of infections acquired during the production process involve, inter alia, feeding or administering health-promoting substances, such as zootechnical feed additives, or therapeutic agents.

The term “administering” or related terms includes oral administration. Oral administration may be via drinking water, oral gavage, aerosol spray or animal feed. The term “zootechnical feed additive” refers to any additive used to affect favorably the performance of animals in good health or used to affect favorably the environment. Examples for zootechnical feed additives are digestibility enhancers, i.e. substances which, when fed to animals, increase the digestibility of the diet, through action on target feed materials; gut flora stabilizers; micro-organisms or other chemically defined substances, which, when fed to animals, have a positive effect on the gut flora; or substances which favorably affect the environment. Preferably, the health-promoting substances are selected from the group consisting of probiotic agents, praebiotic agents, botanicals, organic/fatty acids, zeolithes, bacteriophages and bacteriolytic enzymes or any combinations thereof.

Therapeutic agents are, for example, antibiotics or anti-inflammatory agents.

A further aspect of the present invention is the provision of a method for obtaining a pooled excremental sample being representative for the animal population as a whole, i.e. representing the total pathogen load in an animal population, the method comprising:
(a1) dividing the animal house or the area in which the animal population is kept in a grid pattern of uniform cells;
(a2) identifying at least one random sample collection site within the first cell and taking one first sample at said at least one sample collection site; and
(a3) sequentially collecting individual excremental samples in the remaining cells using the same relative sample collection sites within each cell;
and optionally
(a4) repeating steps (a2) and (a3) for at least one replicate sample.

The required sample size (i.e. the total number of samples to be taken) may be determined using the following formula:

$${n}_{0} = \frac{{Z}^{2} pq}{{e}^{2}}$$

wherein
n₀ is the sample size recommendation
Z is 1.96 for 95% confidence level
p is the estimated portion of the population with the attribute in question q is 1−p, and
e is the confidence interval expressed as decimal.

In general, a minimum of 80 to 100 individual excremental samples are sufficient for most livestock avian populations. As an example, for a broiler flock of 20000 animals, 96 individual samples are required for a confidence level of 95%.

The sample size corresponds to the number of cells in the grid pattern in case one sample is to be taken per cell. In general, in case x samples are to be taken per cell, the sample size is the number of cells, divided by x.

The aforementioned sample collection methods may be used in processes or methods for determining the presence or monitoring the load of at least one specific pathogen in an animal population.

Applications of the methods according to the invention are for example (i) aiding in the diagnosis and/or prognosis of infections acquired during the production process; (ii) monitoring the progress or reoccurrence of these infections or (iii) aiding in the evaluation of treatment efficacy for an animal population undergoing or contemplating treatment.

Applications of the invention in particular help to avoid loss in animal performance like weight gain and feed conversion.

In the following, the invention is illustrated by non-limiting examples and exemplifying embodiments.

### EXAMPLES

C. perfringens was used as exemplary pathogen to be detected in the avian population. netB and cpa serve as a target genes and play a key role in the development of avian necrotic enteritis.

About 20,000 broiler were randomly assigned to broiler houses as part of the normal chicken placement procedures of the company, in accordance to the American Humane Association certified program, which limits density to 6.2 pounds/square foot at slaughter, including substantial management, and auditing needs. All flocks were managed according to company's standard protocols, which are in line with breeder's recommendations for lighting, temperature, and ventilation. Feeds consisted of basal diet (corn and soy) adjusted for birds requirements for starter, grower & finisher feeds. General flock conditions were monitored daily: the availability of feed and water, temperature control, and any unusual conditions. Dead birds were removed and necropsied to determine cause of death and debilitated birds were culled to avoid further suffering.

### Sample Collection

Fecal samples and flock performance data from several standard broiler live production processes were collected daily from days 13 to 24, and days 15 to 22, respectively.

At each collection time point or event, 24 individual samples were picked up from each quadrant of the house with a plastic tong, walking each quadrant in a zig-zag fashion. To avoid cross contamination of samples, new sterile tong was used for each house as well as prescribed biosecurity measures were observed. Furthermore, debris such as wood shavings, litter, etc., were removed from the samples before all samples from the 4 quadrants were composited to form a single pooled sample (consisting of 96 individual fecal samples) in a sterile sample collection bag. The samples were placed an ice and transferred to the laboratory for storage at −80° C.

### DNA Extraction

Each bag with the pooled 96 samples was allowed to thaw slowly at room temperature; then, the feces were transferred into a sterile container and mixed thoroughly with a sterile tongue depressor. Five (5) grams of the homogenized sample were transferred to a proprietary sample collection tube, containing 20 ml of stabilization buffer and glass beads. Fecal samples in the sample collection tubes are stable for up to 7 days at +15° C. to +30° C.

The tube containing the fecal sample was incubated at 70° C. for 20 minutes in a water bath. The tube was then transferred to a Poly Mix Mill (bead beater) for homogenization at 20 Hz for 15 minutes. At the end of the homogenization, the sample was centrifuged at 2000 g for 5 minutes, and 500 μl of the supernatant was used for DNA extraction. DNA extraction was performed with the King Fisher Flex system (Thermo Fisher, USA), adhering to the protocol of Evonik's proprietary fecal extraction kit.

The King Fisher instrument was prepared by uploading a predefined program (“Cper_Extraction_01”) defining the various steps of the extraction process; sampling tips, DNA elution plate, wash plates and sample plate were prepared as described below.

A 96 tips comb was inserted in an empty deep well plate and placed it in the instrument. This was followed by the introduction of 100 μl of the elute buffer in an elution plate and this plate was also placed in the instrument. Furthermore, 500 μl of wash buffers 3, 2 and 1 where place in each well of 3 different wash plates respectively, and these plates were placed on the instrument in the same order. Finally, 300 μl of lysis buffer, 25 μl magnetic beads, 20 μl enhancer, 10 μl internal control and 500 μl of the supernatant from the fecal sample were added to each well of a sample plate. After placing the sample plate on the instrument, the extraction was started by pressing the start button.

### DNA Quantification

For the quantification of markers in the DNA, a 20 μl master mix consisting of 5 μl Master A, 15 μl master B and 1 μl of IC (internal control) was prepared according to the instruction of proprietary Real-Time PCR detection kit of Evonik Nutrition & Care GmbH per reaction. Enough master mix was prepared to accommodate the running of all samples, non-template controls (NTC) and 4 standards (51 to S4) in duplicates. 20 μl of the master mix were dispensed into individual wells of a 96 well plate. Then, a 10 μl of the extracted DNA sample was transferred into each well. 10 μl of the respective standard and 1 μl of IC were transferred to each standard well accordingly. To prepare a NTC, 10 μl of sterile nuclease free water and 1 μl of IC were transferred to the NTC wells each. The contents of the plate were mixed thoroughly with a multi-channel pipet, and the plate was sealed with a Clear Weld Seal Mark II foil. film. The plate was centrifuged for 30 seconds at 1000 g (3000 rpm). Finally, the plate was run on a CFX96 real time PCR instrument (Bio Rad, Germany) with the following PCR conditions: 45 cycles of denaturation at 95° C. for 15 seconds, annealing at 58° C. for 45 seconds and extension at 72° C. for 15 seconds. Data were acquired during the amplification phase of the QPCR run. At the end of the run, data received from the BioRad CFX96 were preprocessed with the Bio-Rad CFX Manager 3.1 and exported to Excel 2013 for further analysis. The quantification of markers in samples were determined from the standard curve constructed with standard solutions (S1 to S4) containing equal concentrations of both targets. The concentrations of netB in S1, S2, S3 and S4 are 10⁴ copies/μl, 10³ copies/μl, 10² copies/μl and 10¹ copies/μl respectively. The log of the standards were plotted along the x-axis, while the Ct (cycle thresholds) were plotted along the y-axis. The resulting linear regression line [y=mx+b or Ct=m (log quantity)+b] was used to determine the concentrations of the targets in the sample tested.

List of Primers and Probe Used for the qPCR to Quantify Levels of Expression of netB:

| | Primers and Probes | | Probe |
|---|---|---|---|
| Target | (where applicable) | | reporter |
| netB | Forward: | 5′-TATACTTCTAGTGATACCGC-3′ | |
| | | (SEQ ID NO.: 1) | |
| | Reverse: | 5′-ATCAGAATGAGGATCTTCAA-3′ | |
| | | (SEQ ID NO.: 2) | |
| | Probe: | 5′TCACACATAAAGGTTGGAAGGCAA | FAM |
| | | C-3′ | |
| | | (SEQ ID NO.: 3) | |

| | | | | Starting | | |
|---|---|---|---|---|---|---|
| | | | mean | quantity for | | |
| Day | Marker | Cq | Cq | 1 g feces | Log10 | Mean |
| 13 | netB | 33.55 | 33.73 | 8.98E+02 | 2.95E+00 | 2.90E+00 |
| | | 33.91 | | 6.97E+02 | 2.84E+00 | |
| 14 | netB | 27.71 | 27.705 | 5.73E+04 | 4.76E+00 | 4.76E+00 |
| | | 27.7 | | 5.77E+04 | 4.76E+00 | |
| 15 | netB | 27.28 | 27.33 | 7.76E+04 | 4.89E+00 | 4.87E+00 |
| | | 27.38 | | 7.22E+04 | 4.86E+00 | |
| 16 | netB | 24.54 | 24.505 | 5.45E+05 | 5.74E+00 | 5.75E+00 |
| | | 24.47 | | 5.72E+05 | 5.76E+00 | |
| 17 | netB | 22.49 | 22.5 | 2.34E+06 | 6.37E+00 | 6.37E+00 |
| | | 22.51 | | 2.30E+06 | 6.36E+00 | |
| 20 | netB | 21.74 | 21.6 | 3.99E+06 | 6.60E+00 | 6.64E+00 |
| | | 21.46 | | 4.87E+06 | 6.69E+00 | |
| 21 | netB | 20.76 | 20.715 | 8.00E+06 | 6.90E+00 | 6.92E+00 |
| | | 20.67 | | 8.50E+06 | 6.93E+00 | |
| 22 | netB | 18.1 | 18.12 | 5.31E+07 | 7.73E+00 | 7.72E+00 |
| | | 18.14 | | 5.15E+07 | 7.71E+00 | |
| 23 | netB | 22.4 | 22.295 | 2.50E+06 | 6.40E+00 | 6.43E+00 |
| | | 22.19 | | 2.89E+06 | 6.46E+00 | |
| 24 | netB | 20.3 | 20.27 | 1.11E+07 | 7.05E+00 | 7.05E+00 |
| | | 20.24 | | 1.16E+07 | 7.06E+00 | |

The outbreak of necrotic enteritis was established by veterinarian diagnosis (necropsy) on day 16.

List of Primers and Probe Used for the qPCR to Quantify Levels of Expression of cpa:

| | Primers and Probes | | Probe |
|---|---|---|---|
| Target | (where applicable) | | reporter |
| cpa | Forward: | 5′-TACATATCAACTAGTGGTGA-3′ | |
| | | (SEQ ID NO.: 4) | |
| | Reverse: | 5′-ATTCTTGAGTTTTTCCATCC-3′ | |
| | | (SEQ ID NO.: 5) | |
| | Probe: | 5′-TGGAACAGATGACTACATGTATT | Cy5 |
| | | TTGG-3 | |
| | | (SEQ ID NO.: 6) | |

| | | | | Starting | | |
|---|---|---|---|---|---|---|
| | | | mean | quantity for | | |
| Day | Marker | Cq | Cq | 1 g feces | Log10 | Mean |
| 15 | cpa | 28.6 | 28.57 | 6.05E+04 | 4.78E+00 | 4.79E+00 |
| | | 28.54 | | 6.26E+04 | 4.80E+00 | |
| 16 | cpa | 26.33 | 26.26 | 290400 | 5.46E+00 | 5.49E+00 |
| | | 26.19 | | 321900 | 5.51E+00 | |
| 17 | cpa | 24.55 | 24.485 | 1.00E+06 | 6.00E+00 | 6.02E+00 |
| | | 24.42 | | 1.10E+06 | 6.04E+00 | |
| 20 | cpa | 24.16 | 23.95 | 1.32E+06 | 6.12E+00 | 6.18E+00 |
| | | 23.74 | | 1.75E+06 | 6.24E+00 | |
| 21 | cpa | 22.7 | 22.665 | 3.61E+06 | 6.56E+00 | 6.57E+00 |
| | | 22.63 | | 3.81E+06 | 6.58E+00 | |
| 22 | cpa | 20.41 | 20.405 | 1.78E+07 | 7.25E+00 | 7.25E+00 |
| | | 20.4 | | 1.79E+07 | 7.25E+00 | |

1-15. (canceled)

16. An in vitro method for monitoring the load of at least one pathogen in an avian population, the method comprising the following steps:
a) collecting and pooling excremental sample material deriving from an avian population;
b) homogenizing the pooled sample material obtained in step a);
c) diluting and optionally stabilizing the pooled sample material obtained in step (b) with aqueous buffer solution;
d) lysing the cell material contained in the diluted sample material obtained in step c);
e) isolating nucleic acid material from the lysed sample material of step d);
f) detecting and quantifying at least one pathogen-specific target gene, or functional fragment thereof, contained in the nucleic acid isolate obtained in step e);
g) repeating steps a) to f) at consecutive points in time; and
h) observing alterations in amount of the at least one pathogen specific target gene over time.

17. The method of claim 16, wherein the avian population is a poultry flock.

18. The method of claim 16, wherein the pathogen is selected from pathogenic bacterial species, pathogenic viral species and/or pathogenic single-cell eukaryotes.

19. The method of claim 16, wherein alterations in the load of more than one pathogen are observed simultaneously.

20. The method of claim 16, wherein the excremental sample material is selected from the group consisting of: litter samples, liquid manure samples, samples of bodily excrements and solutions/suspensions thereof.

21. The method of claim 16, wherein the sample material is feces.

22. The method of claim 16, wherein the pooled sample material obtained in step (a) is a composite sample derived from individual excremental samples.

23. The method of claim 16, wherein the sample size required for the specific population is determined using the following formula:
$${n}_{0} = \frac{{Z}^{2} pq}{{e}^{2}}$$
wherein
n₀ is the sample size recommendation;
Z is 1.96 for 95% confidence level;
p is the estimated portion of the population with the attribute in question q is 1−p; and
e is the confidence interval expressed as decimal.

24. The method of claim 16, wherein the pooled sample material of step (a) is obtained by:
(a1) dividing the animal house or the area in which the animal population is kept in a grid pattern of uniform cells;
(a2) identifying at least one random sample collection site within the first cell and taking one first sample at said at least one sample collection site; and
(a3) sequentially collecting individual excremental samples in the remaining cells using the same relative sample collection sites within each cell; and optionally
(a4) repeating steps (a2) and (a3) for at least one replicate sample.

25. The method of claim 16, wherein the aqueous buffer solution used in step (c) comprises chaotropic salts.

26. The method of claim 16, wherein lysis step (d) includes a heating step (d1), a grinding step (d2) and a spinning step (d3).

27. The method of claim 16, wherein the detection and quantification of the at least one pathogen-specific target gene in step (f) is performed via qPCR.

28. The method of claim 17, wherein the pathogen is selected from pathogenic bacterial species, pathogenic viral species and/or pathogenic single-cell eukaryotes.

29. The method of claim 28, wherein alterations in the load of more than one pathogen are observed simultaneously.

30. The method of claim 28, wherein the excremental sample material is selected from the group consisting of: litter samples, liquid manure samples, samples of bodily excrements and solutions/suspensions thereof.

31. The method of claim 28, wherein the sample material is feces.

32. The method of claim 28, wherein the pooled sample material obtained in step (a) is a composite sample derived from individual excremental samples.

33. The method of claim 28, wherein the sample size required for the specific population is determined using the following formula:
$${n}_{0} = \frac{{Z}^{2} pq}{{e}^{2}}$$
wherein
n₀ is the sample size recommendation;
Z is 1.96 for 95% confidence level;
p is the estimated portion of the population with the attribute in question q is 1−p; and
e is the confidence interval expressed as decimal.

34. The method of claim 33, wherein the pooled sample material of step (a) is obtained by:
(a1) dividing the animal house or the area in which the animal population is kept in a grid pattern of uniform cells;
(a2) identifying at least one random sample collection site within the first cell and taking one first sample at said at least one sample collection site; and
(a3) sequentially collecting individual excremental samples in the remaining cells using the same relative sample collection sites within each cell; and optionally
(a4) repeating steps (a2) and (a3) for at least one replicate sample.

35. The method of claim 34, wherein the aqueous buffer solution used in step (c) comprises chaotropic salts.


SEQUENCE LISTING (6 sequences)

SEQ ID NO: 1 - DNA, 20 bp, Artificial Sequence
  Primer (fwd)
tatacttctagtgataccgc

SEQ ID NO: 2 - DNA, 20 bp, Artificial Sequence
  Primer (rev)
atcagaatgaggatcttcaa

SEQ ID NO: 3 - DNA, 23 bp, Artificial Sequence
  Probe
tcacataaaggttggaaggcaac

SEQ ID NO: 4 - DNA, 20 bp, artificial
  Primer
tacatatcaactagtggtga

SEQ ID NO: 5 - DNA, 20 bp, Artificial Sequence
  Primer
attcttgagtttttccatcc

SEQ ID NO: 6 - DNA, 27 bp, Artificial Sequence
  Probe
tggaacagatgactacatgtattttgg